(19) United States
(12) Patent Application Publication
(10) Pub. No.: US 2023/0363473 A1
(43) Pub. Date: Nov. 16, 2023

(54) SELF-STERILIZING FACE COVERINGS

(71) Applicant: VIRGINIA COMMONWEALTH UNIVERSITY, Richmond, VA (US)

(72) Inventors: Erdem TOPSAKAL, Richmond, VA (US); McKenzie PIPER, Richmond, VA (US); Tania DOBRESCU, Richmond, VA (US); James FORSBERG, Richmond, VA (US); Jonathan LUNDQUIST, Richmond, VA (US)

(21) Appl. No.: 18/245,243

(22) PCT Filed: Sep. 13, 2021
(86) PCT No.: PCT/US2021/050021

§ 371 Date: Mar. 14, 2023

Related U.S. Application Data
(60) Provisional application No. 63/141,688, filed on Jan. 26, 2021.
(60) Provisional application No. 63/077,804, filed on Sep. 14, 2020.

(51) Int. Cl. A41D 13/11; A61L 2/04; A41D 13/005

(52) U.S. Cl. CPC A41D 13/11; A41D 13/0051; A61L 2/04; A61L 2202/14; A61L 2202/26

(57) ABSTRACT

A self-cleaning face covering is provided. The face covering includes a fabric layer having a resisitive heating element disposed on the fabric; and an infrared reflective layer arranged between the fabric layer and an inner surface of the face covering configured to reduce heat transfer to the inner surface. Methods of cleaning the face covering are also provided.

### FIELD OF THE INVENTION

The invention generally relates to face masks having an inkjet or screen-printed heating element for inactivation of viral particles.

### BACKGROUND

The recent SARS-CoV-2 virus outbreak leading to a global pandemic heightened awareness of the need for personal protective articles such as face masks which are now used daily by individuals around the world. Such awareness created a great demand for new products and/or novel approaches that may help to prevent bacterial and/or viral infections. Sanitation and protection by using personal protective equipment (PPE) are commonly used to ensure safety from bacterial and viral infections of broad strains and exposure ranges, some exemplary viruses being SARS-CoV-2 and avian influenza. While total assurance of protection from viruses may not be possible, multiple protocols and protective articles have been developed to enable a safer environment, particularly for shared public places.

Such protective articles are useful to prevent spreading of infections, especially in hospital settings. People at high risk of exposure due to interaction with the public or known infected individuals would benefit from protective articles that could be sanitized during use without the need for frequent replacement. Such individuals include healthcare workers, people who work in areas with a high volume of foot traffic, and public servants such as fire fighters, EMS, and police officers. In addition to minimizing transmission of infections, a self-cleaning protective article would also reduce shortages of PPE and reduce the burden on the environment by creating less waste.

### SUMMARY OF THE INVENTION

The disclosure relates to a protective article, such as a face mask, having an embedded heating element for inactivating microbial pathogens, thus shielding the user from bacterial or viral infections while the mask is being worn.

One aspect of the disclosure provides a face covering comprising a fabric layer having a resisitive heating element disposed on the fabric and an infrared reflective layer arranged between the fabric layer and an inner surface of the face covering configured to reduce heat transfer to the inner surface. In some embodiments, the heating element is an inkjet printed or screen printed heating element. In some embodiments, the heating element provides a sheet resistance of 1-20 Ohms. In some embodiments, the heating element comprises screen printed silver ink. In some embodiments, the heating element is configured to provide a temperature of 60-105° C. to an outer surface of the face covering. In some embodiments, the fabric is substantially formed from cotton. In some embodiments, the infrared reflective layer comprises perforated biaxially-oriented polyethylene terephthalate. In some embodiments, the face covering further comprises a controller configured to regulate a current through the heating element. In some embodiments, the controller uses pulse width modulation at a peak voltage between 3 V and 11 V to control temperature by modifying pulse frequency and/or duty cycle.

Another aspect of the disclosure provides a method of cleaning a face covering comprising providing a face covering as described herein and using the heating element to heat an outside surface of the mask to a temperature of 60-105° C. for a time sufficient to inactivate viral particles. In some embodiments, the method is performed while the face covering is being worn by a user. In some embodiments, the time sufficient to inactivate viral particles is 1-15 minutes.

### BRIEF DESCRIPTION OF THE DRAWINGS

FIG. 1. Diagram of a face covering according to some embodiments of the disclosure.

FIG. 2. Diagram of a controller and power source according to some embodiments of the disclosure.

FIG. 3. A fabric layer with a resistive heating element disposed on the fabric according to some embodiments of the disclosure.

FIG. 4. A full-assembled face covering according to some embodiments of the disclosure.

FIG. 5. A circuit design according to some embodiments of the disclosure.

FIG. 6. A printed circuit board (PCB) design according to some embodiments of the disclosure.

FIG. 7. A power source according to some embodiments of the disclosure.

FIGS. 8A-B. Diagram of a (A) first part and (B) second part of a two-part power source plastic enclosure optimized for mold manufacturing according to some embodiments of the disclosure.

FIG. 9. Bar graph of sheet resistance of majority cotton fabrics.

### DETAILED DESCRIPTION

Embodiments of the disclosure provide a self-cleaning face covering/mask having a heating element configured to heat an outer surface of the mask to inactivate microbial pathogens (e.g. viral particles), thus protecting the wearer from infection.

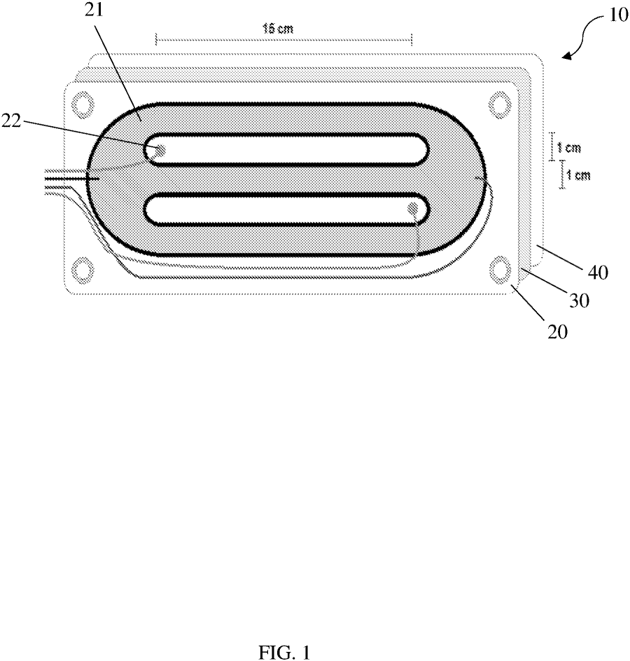

With reference to FIG. 1, the face covering 10 comprises a plurality of layers including a fabric layer 20 having a resisitive heating element 21 disposed on the fabric. Resistive heating elements generate heat by the phenomena of joule heating. As an electrical current passes through the element, heat is generated due to the resistive nature of the element’s design. Resistive heating elements are either composed of metallic alloys, ceramic materials, or ceramic metals. In some embodiments, the heating element is an inkjet printed or screen printed heating element.

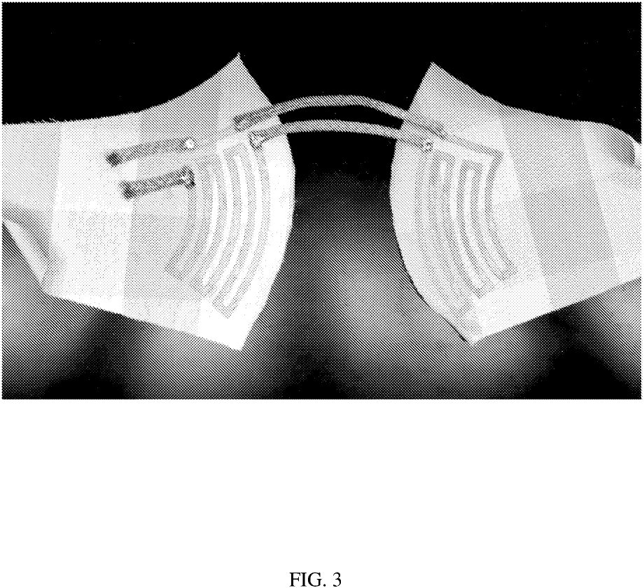

Any conductive ink that provides a sheet resistance of 1-20 Ohms, e.g. about 1.5-10 Ohms, may be used. Suitable inks may contain platinum, silver, silver oxides, gold, copper, and/or aluminum conductive alloys. Suitable inks are commercially available, such as the nano Ag inks from Novacentrix®. Non-metal-containing inks may also be utilized, such as a carbon or nanocarbon ink. A resistive heating element can be made by printing an ink solution onto a fabric. The printing steps are performed to produce printed trails, e.g. forming an interconnected maze-like pattern as shown in FIG. 3. The trails can then be subjected to post-print curing to produce electrically conductive tracks. In some embodiments, a post-print curing step may not be necessary with some ink solutions as it may just need to dry or it may dry immediately upon printing. Possible printers include thermal inkjet printers (e.g., Lexmark etc.), piezoelectric inkjet printers (e.g., Fuki, Dimatix, Epson, Microfab, etc.), aerosol printers (e.g., Optomec), and/or Ultrasonic printers (e.g., SonoPlot). While drop-on-demand dispensing will often prove most economical, continuous dispensing systems are also feasible.

The post-print curing step can involve fusing, sintering, decomposing, and/or firing. The curing step can additionally or alternatively comprise drying, evaporating, or otherwise dismissing substances which are not electrically conductive. The curing steps can instead or further include exposure to radiation (e.g., ultraviolet, pulse light, laser, plasma, microwave etc.), electrical power, or chemical agents.

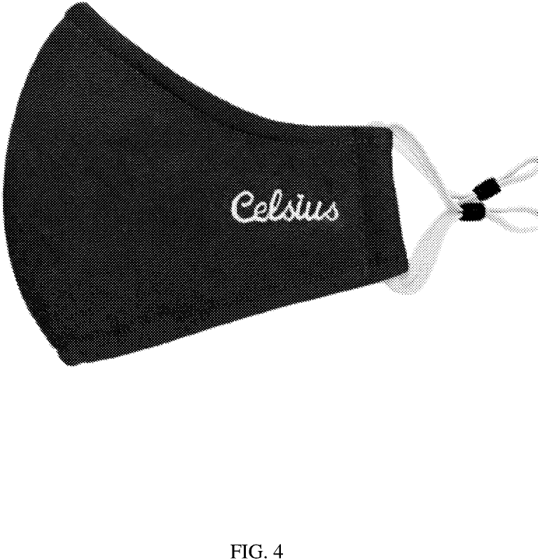

The heating element is configured to heat an outer surface of the face covering to a temperature and for a time sufficient to inactivate microbial pathogens. In some embodiments, the heating element is configured to provide a temperature of at least 60-105° C., e.g. 70-100° C., to an outer surface of the face covering. A temperature sensor 22 (e.g. a thermocouple temperature detector) is provided to monitor the temperature of the heating element. In some embodiments, the heating/cleaning cycle lasts for at least 1-30 minutes, e.g. 1-15 minutes. The “outer surface” of the face covering may be the fabric layer with the heating element or one or more additional fabric layers covering the heating element (FIG. 4).

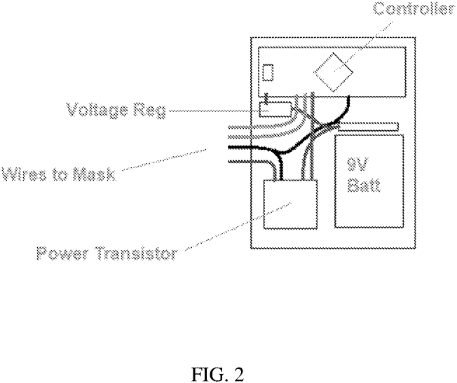

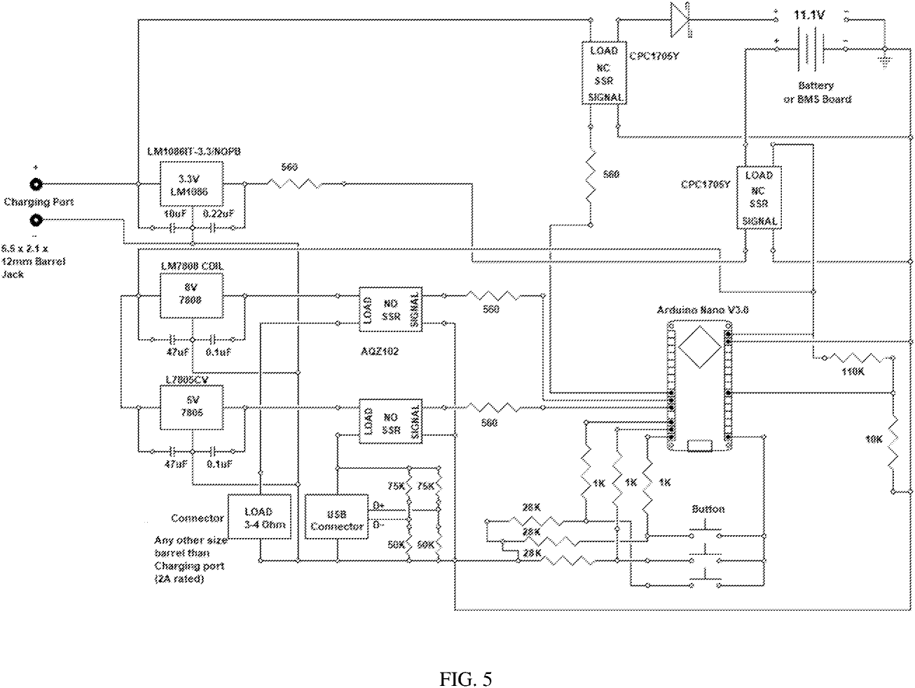

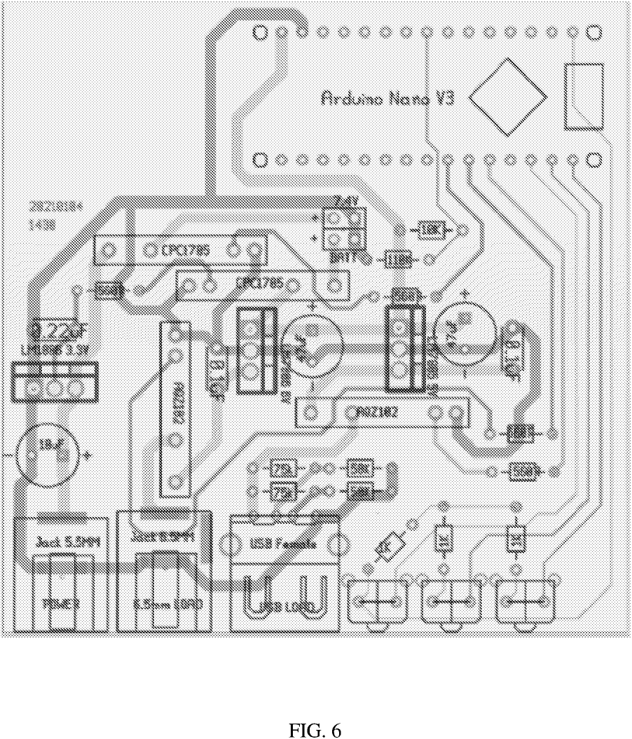

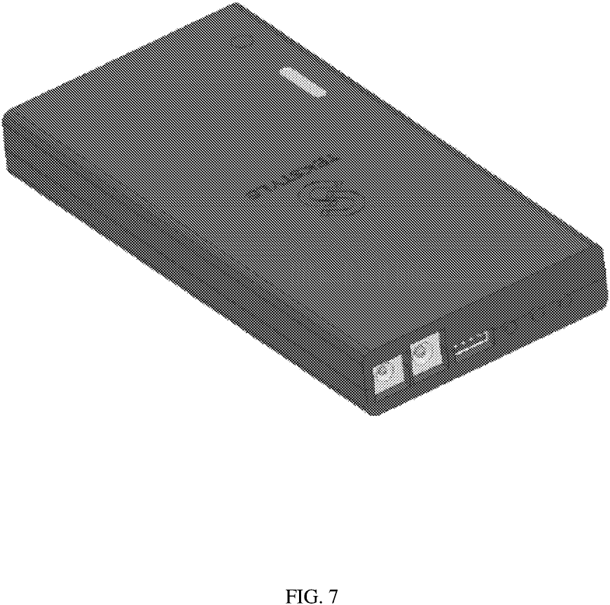

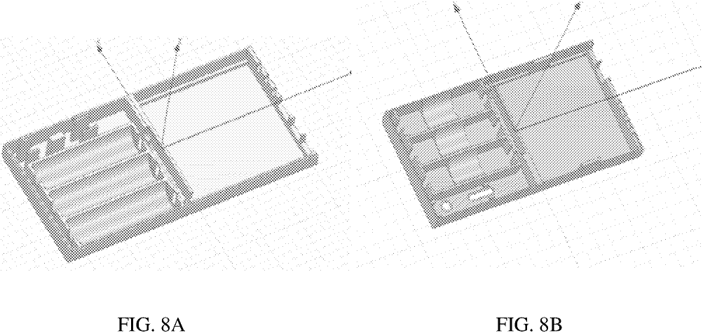

With reference to FIG. 2, the heating element is operably connected, e.g. via wires, to a controller and power source (e.g. a battery). The controller may be used to automatically, e.g. at predetermined times, and/or manually trigger a heating/cleaning cycle. The controller regulates current through the heating element to control temperature, or may control temperature through a feedback temperature probe. The controller may include an electronic circuit, e.g. as shown in FIG. 5 or as a printed circuit board (PCB) as shown in FIG. 6. The controller may be attached behind the head, or placed on a lanyard around the neck, or attached to clothing, or placed in a pocket, or placed on the body in any other way. In some embodiments, the controller may use pulse width modulation at a peak voltage between 3 V and 11 V to control temperature by modifying pulse frequency and/or duty cycle as appropriate or a DC voltage control of 7.4 v or 11.1 v with a 2A current limiter. An exemplary power source is shown in FIG. 7 and an exemplary two-part power source plastic enclosure optimized for mold manufacturing is shown in FIGS. 8A-B. For the heating element, any material and ink paring that produces a sheet resistance of 1-20 Ohms per square can be used with appropriate adjustment of power source (i.e. battery voltage, current and/or pulse peak/duration).

In some embodiments, the power source used to power the heating of the mask may also be used to power other portable electronic devices such as cell phones tablets, or other USB 5V sources, while the mask is in use.

As shown in FIG. 1, the face covering also includes an infrared reflective layer 30 arranged between the fabric layer and an inner surface of the face covering configured to reduce heat transfer to the inner surface, thus protecting the wearer’s face while the heating/cleaning cycle is activated. In some embodiments, the infrared reflective layer comprises perforated biaxially-oriented polyethylene terephthalate (i.e. Mylar®). Other suitable materials include polyaniline polymers and other IR reflective polymers. The face covering may further comprise one or more additional fabric layers 40 arranged between the infrared reflective layer and the inner surface, thus providing a smooth layer contacting the wearer’s face to improve comfort. One or more additional fabric layers may also be provided between the heating element layer and the infrared reflective layer.

The fabric layer with embedded heating element described herein may also be incorporated into masks and respirators known in the art. Masks and respirators contemplated within the scope of the present disclosure include both disposable and non-disposable masks and respirators, and include masks and respirators, and filter materials thereof, that can be reused and washed. A face mask or respirator of the disclosure includes those that cover a wearer’s nose and/or mouth, and even preferably, a portion of the wearer’s face, i.e., cheeks, jaw, chin, and so forth. In one embodiment, a respirator is an N-95, N-99, N-100, R-95, R-99, R-I00, P-95, P-99, or P-I00 respirator. Suitable masks or respirators may also include a bendable metal reinforcement nose bar to allow custom fitting of the mask around the nose of a wearer. Suitable masks may utilize ear loops, e.g. as shown in FIG. 4, or may have straps that are configured to wrap around a wearer’s head.

The fibers contemplated for use in a face covering as described herein can be made of natural materials, such as cotton, paper, and the like, and/or synthetic materials, such as polyethylene, polyester, nylon, rayon, and the like. In some embodiments, the fabric layer containing the heating element is substantially formed from cotton, e.g. at least about 50% cotton, e.g. at least about, 60%, 70%, 80%, 85%, 90%, 95%, 96%, 97%, 98%, or 99% cotton by weight. Additional suitable fibers include but are not limited to: kapok, flax, ramie, kenaf, abaca, coir, hemp, jute, sisal, bamboo fiber, Tencel® and Modal® fibers, glass fibers, basalt fibers, Kevlar® fibers, aramid fibers, wool (which may be obtained, for example, from one of the forty or more different breeds of sheep, and which currently exists in about two hundred types of varying grades), silk, acetate, acrylic, triacetate, spandex (an elastomeric man-made fiber such as Lycra®), polyolefin/polypropylene (man-made olefin fibers), microfibers and microdeniers, lyocell, vegetable fiber (a textile fiber of vegetable origin, such as cotton, kapok, jute, ramie, polylactic acid (PLA) or flax), vinyl fiber, alpaca, angora, and carbon fiber. The fibers can be woven into any suitable material or fabric, or they can be spun-bonded, melt blown, pressed, matted, glued, or otherwise formed into a material or fabric.

In another embodiment, the one or more of the fabric layers may include additional anti-infection agents. In these embodiments, any antimicrobial agent, such as an antibacterial agent, an antibiotic agent, an antiseptic agent, etc., may be used to prevent infection. As used herein, the term “antimicrobial” refers to killing activity or inhibition or prevention of growth or replication of virus, bacteria, fungi, yeast, mold, and mildew. Viruses that can be killed or inhibited by the present invention include, but are not limited to, SARS virus (e.g. SARS-CoV-2), influenza virus (including avian influenza), filoviruses (Marburg and Ebola), hantavirus, and mv virus. Bacteria that can be killed or inhibited by the present invention include, but are not limited to, Bacillus cereus, Bacillus thuringiensis, Mycobacterium tuberculosis, Legionella pneumophila, Escherichia coli, Klebsiella Pneumoniae, Salmonella gallinarum, Salmonella typhimurium, P. gingivalis, Staphylococcus aureus, Staphylococcus epidermis, Streptococcus faecalis, Streptococcus agalactiae, Streptococcus Pseudomonas aeruginosa, Listeria mutans. monocytogenes, Proteus mirabilis, Proteus vulgaris, Vibrio parahaemolyticus, Enterobacter aerogines, Trichophyton malmsten, and Chaetomium globosum. Yeast and mold that can be killed or inhibited by the present invention include, but are not limited to, Stachybotrys. Aspergillus niger, Candida albicans, Pencillium funiculosum, Gliocladum virens, Aureobasidium pullulans, and Saccharomyces cerevisiae.

Further embodiments of the disclosure provide a method of cleaning a face covering comprising providing a face covering as described herein and using the heating element to heat an outside surface of the mask to a temperature and a time sufficient to inactivate microbial pathogens such as viral particles. In some embodiments, the method is performed while the face covering is being worn by a user.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE

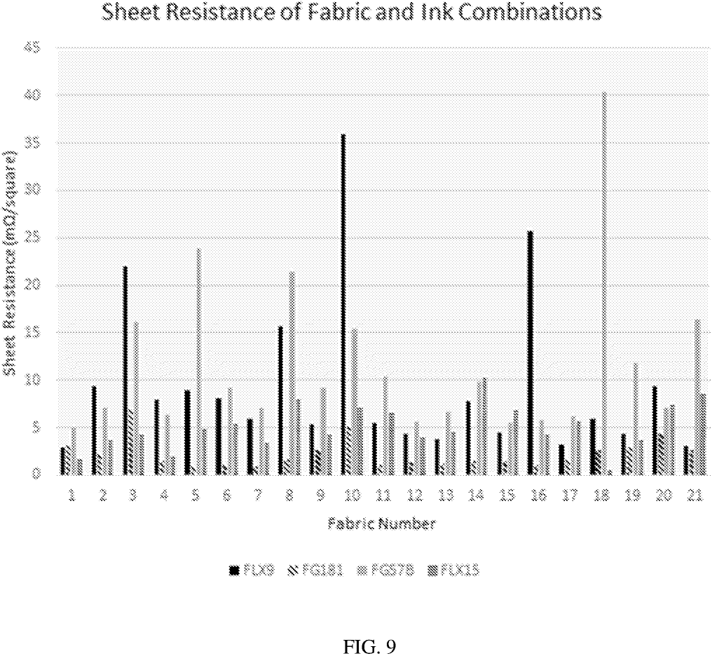

A variety of different fabric compositions were tested in combination with a screen printed silver ink composition as the heating element (Table 1). The sheet resistance for selected fabrics is shown in FIG. 9.

TABLE 1

Fabric compositions

| Fabric No. | Fabric Conten t (%) | | | | | | | | | | |
|---|---|---|---|---|---|---|---|---|---|---|---|
| | Viscos e | Silk | Cotton | Spande x | Rayon | Nylon | Polyest er | Acrylic | Lyocell | Linen | Wool |
| 1 | 80 | 20 | 0 | 0 | 0 | 0 | 0 | 0 | 0 | 0 | 0 |
| 2 | 0 | 0 | 97 | 3 | 0 | 0 | 0 | 0 | 0 | 0 | 0 |
| 3 | 0 | 0 | 5 | 0 | 4 | 5 | 38 | 10 | 0 | 0 | 38 |
| 4 | 0 | 0 | 100 | 0 | 0 | 0 | 0 | 0 | 0 | 0 | 0 |
| 5 | 0 | 0 | 100 | 0 | 0 | 0 | 0 | 0 | 0 | 0 | 0 |
| 6 | 0 | 100 | 0 | 0 | 0 | 0 | 0 | 0 | 0 | 0 | 0 |
| 7 | 0 | 0 | 100 | 0 | 0 | 0 | 0 | 0 | 0 | 0 | 0 |
| 8 | 0 | 35 | 0 | 0 | 65 | 0 | 0 | 0 | 0 | 0 | 0 |
| 9 | 0 | 58 | 0 | 0 | 0 | 42 | 0 | 0 | 0 | 0 | 0 |
| 10 | 0 | 100 | 0 | 0 | 0 | 0 | 0 | 0 | 0 | 0 | 0 |
| 11 | 40 | 0 | 60 | 0 | 0 | 0 | 0 | 0 | 0 | 0 | 0 |
| 12 | 0 | 0 | 60 | 0 | 0 | 0 | 40 | 0 | 0 | 0 | 0 |
| 13 | 0 | 0 | 100 | 0 | 0 | 0 | 0 | 0 | 0 | 0 | 0 |
| 14 | 0 | 0 | 100 | 0 | 0 | 0 | 0 | 0 | 0 | 0 | 0 |
| 15 | 0 | 0 | 10 | 0 | 16 | 0 | 0 | 0 | 49 | 25 | 0 |
| 16 | 0 | 100 | 0 | 0 | 0 | 0 | 0 | 0 | 0 | 0 | 0 |
| 17 | 0 | 65 | 0 | 0 | 0 | 35 | 0 | 0 | 0 | 0 | 0 |
| 18 | 0 | 0 | 0 | 0 | 0 | 0 | 0 | 0 | 0 | 0 | 100 |
| 19 | 0 | 0 | 100 | 0 | 0 | 0 | 0 | 0 | 0 | 0 | 0 |
| 20 | 0 | 0 | 0 | 0 | 45 | 0 | 0 | 0 | 0 | 55 | 0 |
| 21 | 0 | 0 | 100 | 0 | 0 | 0 | 0 | 0 | 0 | 0 | 0 |

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A face covering, comprising:
a fabric layer having a resisitive heating element disposed on the fabric; and
an infrared reflective layer arranged between the fabric layer and an inner surface of the face covering configured to reduce heat transfer to the inner surface.

2. The face covering of claim 1, wherein the heating element is an inkjet printed or screen printed heating element.

3. The face covering of claim 2, wherein the heating element provides a sheet resistance of 1-20 Ohms.

4. The face covering of claim 2, wherein the heating element comprises screen printed silver ink.

5. The face covering of claim 1, wherein the heating element is configured to provide a temperature of 60-105° C. to an outer surface of the face covering.

6. The face covering of claim 1, wherein the fabric is substantially formed from cotton.

7. The face covering of claim 1, wherein the infrared reflective layer comprises perforated biaxially-oriented polyethylene terephthalate.

8. The face covering of claim 1, further comprising a controller configured to regulate a current through the heating element.

9. The face covering of claim 8, wherein the controller uses pulse width modulation at a peak voltage between 3 V and 11 V to control temperature by modifying pulse frequency and/or duty cycle.

10. A method of cleaning a face covering, comprising:
providing the face covering of claim 1; and
using the heating element to heat an outside surface of the mask to a temperature of 60-105° C. for a time sufficient to inactivate viral particles.

11. The method of claim 10, wherein the method is performed while the face covering is being worn by a user.

12. The method of claim 10, wherein the time sufficient to inactivate viral particles is 1-15 minutes.
